(19) United States
(12) Patent Application Publication
(10) Pub. No.: US 2018/0317847 A1
(43) Pub. Date: Nov. 8, 2018

(54) MEDICAL DEVICE AND METHOD OF USING

(71) Applicant: Frank J Ripperger, III, WOODBURY, CT (US)

(72) Inventor: Frank J Ripperger, III, WOODBURY, CT (US)

(21) Appl. No.: 15/972,064

(22) Filed: May 4, 2018

Related U.S. Application Data
(60) Provisional application No. 62/501,654, filed on May 4, 2017.

(51) Int. Cl. A61B 5/00; A61F 5/01; A43B 3/00

(52) U.S. Cl. CPC A61B 5/6812; A61F 5/0102; A61B 5/1036; A61F 2005/0165; A43B 3/0005

(57) ABSTRACT

A medical treatment device having a housing releasably engagable with a portion of a living organism comprising a force sensor in electronic communication with an electronic processing unit and a power source; wherein the electronic processing unit is in electronic communication with an indication device, wherein the electronic processing unit is capable of electronic communication with the indication device, or a combination thereof. Methods of using the device are also disclosed.

### RELATED APPLICATIONS

This application claims priority benefit to U.S. Provisional Application No. 62/501,654, filed May 4, 2017, the disclosures of which are fully incorporated herein by reference.

### BACKGROUND

To assist with healing and/or to prevent injury or additional injury to a living organism, for example a human limb, a clinician may request an individual or therapist to enforce a weight bearing restriction on the limb thereby reducing the amount of force or pressure (e.g., weight) placed on the limb to protect it from injury and/or to facilitate correct healing. For example, a clinician may request a maximum force on a broken leg of 25% of the individual's weight of 100 pounds. Accordingly, the individual is requested to place no more than 25 pounds of force on the limb. Enforcing such requests is problematic.

There remains a need in the art for a portable or medical treatment device which enables one to measure the amount of force applied to a specific body part, to determine a total amount of force applied over time, and/or to determine if a force applied to a limb exceeds a predetermined maximum amount of force or an amount of force considered to be safe, in real time throughout the activities of the individual over a period of time.

### SUMMARY

This summary provides a general overview of embodiments disclosed herein, which are further disclosed herein. This summary is not intended to identify key or essential features of embodiments disclosed herein, nor intended to limit the scope of the instant application, which is described in the description and claims disclosed and recited herein. The instant disclosure relates generally to medical treatment device, more particularly to a medical treatment device capable of providing information relative to the amount of force (e.g., weight) being put on a limb to the wearer of the device and/or to a clinician, which in embodiments includes providing notification when an amount of force applied to a limb exceeds a predetermined set point of the device. In embodiments the device may also function to record instances and amounts of force applied to a limb or other bodily part over a period of time.

In embodiments, a medical treatment device comprises a housing releasably engagable with a portion of a living organism comprising a force sensor in electronic communication with an electronic processing unit and a power source; wherein the electronic processing unit is in electronic communication with an indication device, wherein the electronic processing unit is capable of electronic communication with the indication device, or a combination thereof.

In embodiments, a method of medical treatment comprises the steps of providing a medical treatment device comprising a housing releasably engagable with the portion of a living organism and comprising a force sensor in electronic communication with an electronic processing unit and a power source, wherein the electronic processing unit is in electronic communication with, and/or is capable of electronic communication with an indication device; wherein the electronic processing unit is programmable to accept one or more predetermined force setpoints, and/or one or more ranges of force, and wherein the electronic processing unit actuates the indication device to provide a first visual indication, a first audible indication, a first haptic indication, or a combination thereof, when an amount of force applied to the force sensor exceeds a first force setpoint, is within a first predetermined range of force, or a combination thereof; programming the electronic processing unit with one or more predetermined force setpoints, and/or one or more ranges of force, and engaging the medical treatment device with a portion of a living organism.

### BRIEF DESCRIPTION OF THE SEVERAL VIEWS OF THE DRAWINGS

Embodiments of medical treatment device are described with reference to the following figures. The same numbers are used throughout the figures to reference like features and components.

FIG. 1 is a block diagram of an embodiment of a medical treatment device according to the instant disclosure.

FIG. 2 is a side view of the medical device shown in FIG. 1;

FIG. 3 is a perspective view of a force sensor according to embodiments of the instant disclosure;

FIG. 4 is a perspective view of an alternative embodiment of the medical treatment device according to the instant disclosure;

FIG. 5 is a block diagram view of circuit board utilized in the embodiment shown in FIG. 4;

FIG. 6 is a top block diagram view of an embodiment of the medical device disclosed herein having a plurality of force sensors;

FIG. 7 is a side view of an embodiment of the medical device disposed within an orthopedic device;

FIG. 8 is a top view of FIG. 7;

FIG. 9 is an embodiment of the medical device disposed within an orthopedic walking boot;

FIG. 10 shows the embodiment shown in FIG. 9 disposed on a human foot;

FIG. 11 is an embodiment of the medical device integral to a surgical shoe and disposed about a human foot in a plaster cast;

FIG. 12 is an embodiment of the medical device integral to a fiberglass cast disposed about a human foot; and

FIG. 13 is an embodiment of the medical device arranged to detect and/or prevent pressure sores.

### DETAILED DESCRIPTION

At the outset, it should be noted that in the development of any such actual embodiment, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure. In addition, the device used/disclosed herein can also comprise components other than those cited. In the summary and this detailed description, each numerical value should be read once as modified by the term “about” (unless already expressly so modified), and then read again as not so modified unless otherwise indicated in context.

The following definitions are provided in order to aid those skilled in the art in understanding the detailed description.

As used in the specification and claims, “near” is inclusive of “at.” Reference to a body part may include any portion of living organism or creature. Reference to a “limb” may include an entire limb e.g., an arm, a leg, a head, a foot, a hand, or any portion thereof. For purposes herein, except where specifically indicated otherwise, reference to a body part and to a limb are used interchangeably. Although the description is directed to a human being, it is to be understood that in embodiments, the body part and/or limb may belong to an animal, e.g., a dog or a cat, i.e., a living organism.

For purposes herein, a force sensor may be any electronic device or assembly which produces an electronic response when acted on by an external force. Force may refer to weight and may also refer to any other force which may act upon a body part.

For purposes herein, an electronic processing unit refers to any circuit or collection of circuits, integrated circuits, and electronic components necessary for the unit to function as a digital computing device consistent with description provided herein. For purposes herein, an electronic processing unit may include any number of sub-units as may be required for the unit to function and communicate with other electronic devices including non-volatile memory for retrieval of data stored for subsequent retrieval.

For purposes herein, a power source may be a battery, an inductor, and/or the like capable of powering the circuitry of the force sensor, the electronic processing unit, and the like. As used herein, a visual indication device refers to any device capable of being actuated to produce light e.g., an LED, an alpha-numeric display screen, and the like. Examples include the circuitry and programming required to transmit data to a portable electronic device such as a smart phone, which in-turn produces a visual indication on the viewing screen of the device. For purposes herein, an audible indication device refers to any device capable of making an audible noise. Examples include a speaker, a piezo electric device, the circuitry and programming required to transmit data to a portable electronic device such as a smart phone, which in-turn produces an audible signal, and/or the like. A haptic indication device refers to any device capable of producing a tactile sensation, such a vibration. Examples include vibrators, buzzers, the circuitry and programming required to transmit data to a portable electronic device such as a smart phone, which in-turn produces a vibratory signal, and/or the like.

As used herein, the term “and/or” refers to both the inclusive “and” case and the exclusive “or” case, whereas the term “and or” refers to the inclusive “and” case only and such terms are used herein for brevity. For example, a component comprising “A and/or B” may comprise A alone, B alone, or both A and B; and a component comprising “A and or B” may comprise A alone, or both A and B.

For purposes herein, a dedicated electronic device is a purpose-built device suitable for a limited purpose, as contrasted with a portable electronic device which is suitable for a number of purposes. Examples of portable electronic devices include laptop computers, smart phones, tablets, and the like.

As used herein, an electronic device or unit which is in electronic communication with another electronic unit refers to one way and/or two-way digital communication between the two electronic units. Reference to an electronic device or unit which is in capable of electronic communication with another electronic unit refers to electronic units which have the necessary electronic components, programming and the like for one way and/or two-way digital communication with at least one additional electronic unit, but which are not necessarily in constant electronic communication with the other electronic units. For example, a first electronic unit in wired contact with a second electronic unit may be in constant electronic communication with that second electronic unit. A first electronic unit equipped for wireless electronic communication with a second electronic unit so equipped, e.g., via radio such as Bluetooth, and/or the like, may not necessarily be in constant electronic communication, but for purposes herein the first electronic unit is considered to be capable of electronic communication with the other electronic device.

For purposes herein, an orthopedic device refers to a medical device directed to correction of deformities or functional impairments of the skeletal system, especially the extremities and the spine, and associated structures, as muscles and ligaments. An orthopedic brace refers to a medical device designed to address musculoskeletal issues, which includes those used to properly align, correct the position, support, stabilize, and protect certain parts of the body (particularly the muscles, joints, and bones) as they heal from injury or trauma.

In addition, an orthopedic device may include crutches, wheelchairs, and even a bed when employed as part of a medical treatment. A cast refers to the various types of protective shells of fiberglass, plastic, or plaster, and bandage that are molded to protect a broken or fractured limb as it heals. An orthopedic implant refers to an orthopedic device manufactured to replace a missing orthopedic structure, support a damaged orthopedic structure, or enhance an existing orthopedic structure. For purposes herein, a prosthesis refers to an artificial device that replaces a missing body part, which may be lost through trauma, disease, or congenital conditions, and which is intended to restore the normal functions of the missing body part.

For purposes herein pressure sores, also referred to bedsores, pressure ulcers, decubitus ulcers, and the like refer to injuries to skin and underlying tissue resulting from prolonged pressure on the skin. These include sores which develop on skin that covers bony areas of the body, such as the heels, ankles, hips, tailbone, and those in contact with orthopedic devices.

For purposes herein, engagement with a portion of a living organism refers to contact which allows force to be transmitted through an object eventually to the living organism. Direct contact, e.g., device to skin, is not required for purposes herein. Accordingly, an example of the medical device according to the instant disclosure which is releasably engaged with a portion of living organism includes the medical device located on the bottom of a shoe worn on a human foot. For purposes herein, the medical device is considered to be engaged with the foot even though the medical device is not in direct physical contact with the foot.

In embodiments, a medical treatment device comprises a housing releasably engagable with a portion of a living organism comprising a force sensor in electronic communication with an electronic processing unit and a power source; wherein the electronic processing unit is in electronic communication with an indication device, wherein the electronic processing unit is capable of electronic communication with the indication device, or a combination thereof.

In embodiments, the electronic processing unit is programmable to accept one or more predetermined force setpoints, and/or one or more ranges of force, and wherein the electronic processing unit actuates the indication device to provide a first visual indication, a first audible indication, a first haptic indication, or a combination thereof, when an amount of force applied to the force sensor exceeds a first force setpoint, is within a first predetermined range of force, or a combination thereof, and optionally to actuate the indication device to provide a second visual indication, a second audible indication, a second haptic indication, or a combination thereof, when the amount of force applied to the force sensor exceeds a second force setpoint, is within a second predetermined range of force, or a combination thereof.

In embodiments the electronic processing unit includes an electronic storage which records the force applied to the force sensor in the form of digital data, and wherein these data are retrievable from the electronic storage by an external data retrieval device placed in electronic communication with the electronic processing unit. In embodiments the device may further comprise a temperature sensor, an acceleration sensor, a clock, or a combination thereof in electronic communication with the electronic processing unit, and wherein the electronic processing unit records the temperature, time, acceleration force, or a combination thereof in the form of digital data, and wherein these data are retrievable from the electronic storage by the external data retrieval device placed in electronic communication with the electronic processing unit.

In embodiments the indication device is located on the housing, is a dedicated electronic device in wireless communication with the electronic processing unit, or a combination thereof. In other embodiments the indication device comprises a program running on a portable electronic device, wherein the electronic processing unit is in, or is capable of being placed in wired electronic communication, wireless electronic communication, or intermittent wireless electronic communication with the portable electronic device, which in embodiments is a cell phone, typically referred to as a smart phone, a tablet, or another programmable, general purpose hand-held computing device.

In embodiments the medical treatment device may comprise a plurality of force sensors. In embodiments each of the force sensors is in electronic communication with a single electronic processing unit, while in alternative embodiments each of the force sensors is in electronic communication with one or more electronic processing units.

In embodiments the housing comprises one or more attachment straps dimensioned and arranged to engage the housing with the portion of the living organism.

In embodiments the housing is integral to, or dimensioned and arranged to be disposed on or within an orthopedic device. In embodiments the orthopedic device is selected from the group consisting of: a removable orthopedic brace, a removable orthopedic cast, an orthopedic brace, a plaster cast, a synthetic cast, a splint, an implant, a wheel chair, crutches, a prosthesis, and a combination thereof.

In embodiments, the housing is disposed within or is integral to a foot covering, and wherein the force sensor is arranged to measure the force applied to the foot upon contact of an outer portion of the foot covering with an external surface.

In embodiments the housing is disposed within, or is integral to a hand covering, a wrist covering, or a combination thereof, and wherein the force sensor is arranged to measure the force applied to the hand, the wrist, or a combination thereof upon contact of an outer portion of the covering with an external surface.

In embodiments the housing is disposed between the portion of the living organism, e.g., a human being, and an external surface, e.g., a rub point on a cast, a bed, or a wheelchair, wherein the location of the force sensor, the pressure set points and/or pressures ranges are selected to measure the force applied to the portion of the living organism consistent with the formation of pressure sores, pressure ulcers, bed sores, or a combination thereof.

In embodiments, a method of medical treatment comprising the steps of providing a medical device according to one or more embodiments disclosed herein, programming the electronic processing unit with one or more predetermined force setpoints, and/or one or more ranges of force, and engaging the medical treatment device with a portion of a living organism.

In embodiments wherein the electronic processing unit includes an electronic storage which records the force applied to the force sensor in the form of digital data, and wherein these data are retrievable from the electronic storage by an external data retrieval device in electronic communication with the electronic processing unit, the method further comprises recording the force applied to the force sensor over a period of time in the form of digital data, and optionally electronically retrieving these data from the electronic storage using the external data retrieval device.

In embodiments wherein the indication device comprises a program running on a portable electronic device, the method further comprises placing the electronic processing unit in electronic communication with the portable electronic device comprising the indication device.

In embodiments, the method includes selecting the location of the housing such that it is disposed between the portion of the living organism and an external surface, and the force sensor is arranged to measure the force applied to the portion of the living organism consistent with the formation of pressure sores, pressure ulcers, bed sores, or a combination thereof, and wherein the one or more predetermined force setpoints, and/or the one or more ranges of force are selected to be in a range known to form pressure sores, pressure ulcers, bed sores, or a combination thereof.

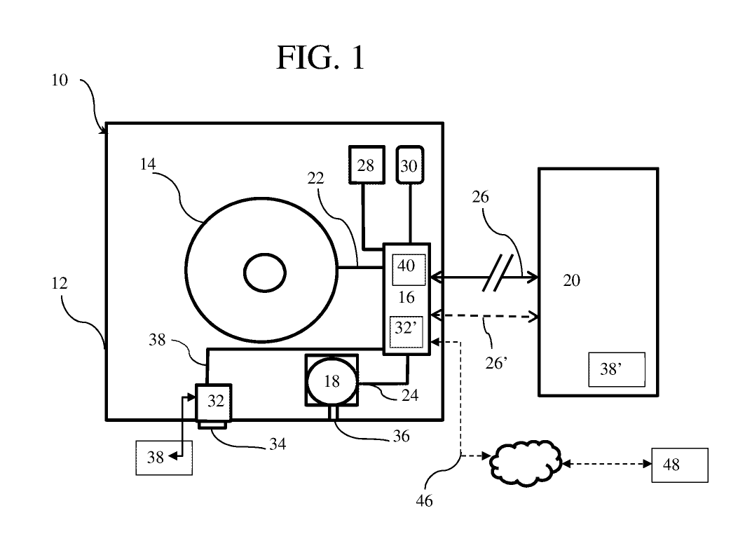

Turning now to the figures, in which similar reference characters denote similar elements throughout the several views, as shown in FIG. 1, in embodiments the medical treatment device, generally referred to as 10, comprises a housing 12 releasably engagable with a portion of a living organism comprising a force sensor 14 in electronic communication 22 with an electronic processing unit 16 and in electronic communication with a power source 18 via connector 24. The electronic processing unit 16 is in electronic communication 26 with an indication device 20. The electrical communication 26 may be wired communication, wireless communication 26′, or any combination thereof.

In embodiments, the medical treatment device 10 may further include additional sensors such as a temperature sensor 28, an acceleration sensor 30, a clock 40, and the like in electronic communication with the electronic processing unit 16.

In embodiments, the medical device 10 may further include a digital electronic memory component 32 in electronic communication 38 with the electronic processing unit 16.

The digital electronic memory component 32 being configured to record, e.g., log, digital data regarding forces applied, time, and/or inputs from other sensors such as temperature and/or acceleration force from sensors 28 and/or 30. In embodiments, the digital electronic memory component 32 may be integral to the electronic processing unit 16, shown in FIG. 1 as 32′. In addition, or in an alternative embodiment, the electronic memory may be separate from the electronic processing unit 16, and/or may include a removable memory storage unit 34, e.g., an SD memory card, a USB memory drive, and/or other digital memory devices commonly known in the art. Accordingly, in embodiments of the medical treatment device, the electronic processing unit includes an electronic storage which records the force applied to the force sensor in the form of digital data.

These data are retrievable from the digital electronic memory component 32 by an external data retrieval device 38. Examples include removal of the removable memory storage unit 34 from the medical device 10 with subsequent electronic communication with an external data retrieval device, e.g., a computer (not shown) and/or via wired and/or wireless communication with an external data retrieval device 38 which is placed in electronic communication with the electronic processing unit 16, directly with the digital electronic memory component 32, or any combination thereof. In embodiments, the external data retrieval device 38 may be included with the indication device 20, shown in FIG. 1 as 38′. In embodiments, the electronic processing unit 16 may be in wireless contact with one or more data collecting, data storage, and/or monitoring functions via wireless communication 46, e.g., via Wi-Fi or Bluetooth, and may be configurable via wireless electronic communication 46, and/or may provide an indication utilizing a wireless device, e.g., a smartphone or networked device 48.

In embodiments, the power source 18 may be a battery, an inductor, and/or any power source capable of providing the necessary power to the electronic circuitry to function according to the instant disclosure. As shown in FIG. 1, the power source 18 may be a rechargeable battery, in electrical communication with an external charging port 36 of any type common in the art.

In embodiments the electronic processing unit 16 is programmable to accept one or more predetermined force setpoints, and/or one or more ranges of force, and wherein the electronic processing unit actuates the indication device to provide a first visual indication, a first audible indication, a first haptic indication, or a combination thereof, when an amount of force applied to the force sensor exceeds a first force setpoint, is within a first predetermined range of force, or a combination thereof; and optionally to actuate the indication device to provide a second visual indication, a second audible indication, a second haptic indication, or a combination thereof, when the amount of force applied to the force sensor exceeds a second force setpoint, is within a second predetermined range of force, or a combination thereof. The electronic processing unit 16 may be programmed via the indication device, by an external device and/or the like. In embodiments, different indications may be assigned to different force setpoints and/or different force ranges. For example, the indication device may display a green visual signal when the force applied is below a first selected value, a yellow signal when above the first selected value but below a second selected value, and a red indication when the force is above the second selected value.

The visual indications may include color changes to the indication device, illumination of LEDs on the indication device, a numeric display on the indication device, and/or any combination thereof. Audible indications may be a buzzer, a ringtone, and/or the like. In embodiments, the volume of the audible indication may be proportional to the amount of force being applied to the sensor and thus to the limb of the wearer. Different audible tones may be associated with various setpoint and/or force ranges. Haptic indications may be a vibrator, a buzzer, and/or the like. In embodiments, the intensity of the haptic indication may be proportional to the amount of force being applied to the sensor and thus to the limb of the wearer. Different haptic indicators may be associated with various setpoint and/or force ranges.

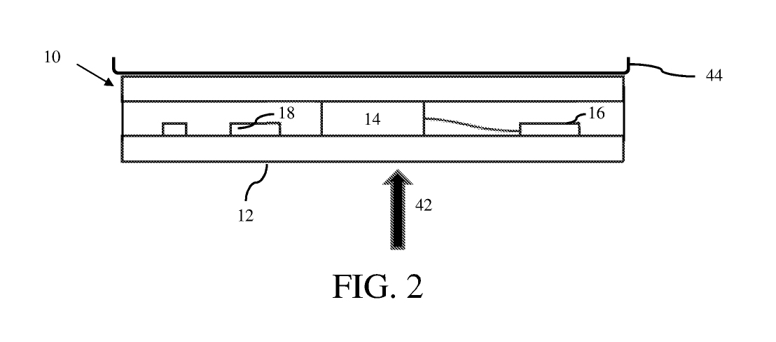

FIG. 2 shows a side view of a medical treatment device according to the instant disclosure. As shown in FIG. 2, the force sensor 14 may be disposed within housing 12 such that force acting on housing 12, indicated by arrow 42, is transferred through the device to the portion of the limb or appendage to which the medical device is engaged 44.

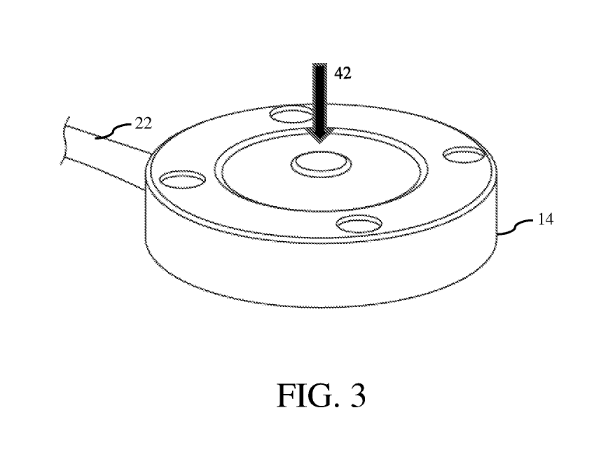

FIG. 3 shows a force sensor 14 according to embodiments of the instant disclosure. A force 42 acting on the force sensor results in an electronic signal proportional to the force applied.

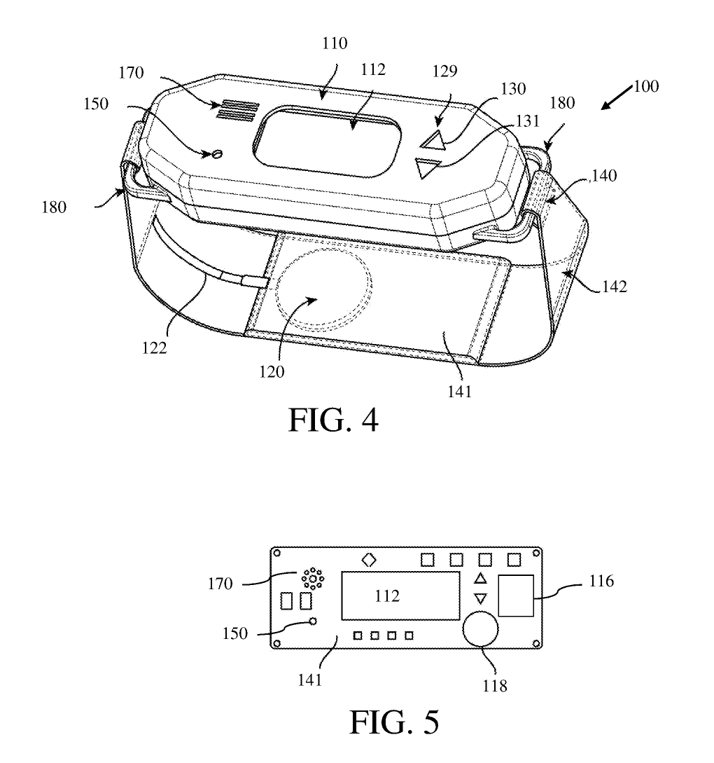

As shown in FIG. 4, in an embodiment the medical device is releasably engageable about an appendage such as the human hand or foot (not shown). The medical treatment device, generally represented as 100 includes a body 110 comprising a rigid portion 180 comprising a plurality of strap anchors 140 disposed and connected to the body at each end engaged by a flexible, adjustable encasement strap 142 attached to each side. In embodiments, the force sensor, e.g., a force sensor 120 is disposed within or on the adjustable encasement strap 141 and in electrical communication with the electronic processing unit via conductor 122. The force sensor 120 senses the force applied to a limb (not shown) when disposed between a portion of the limb and a point of contact of a surface exerting a force on the limb. For example, the force sensor may be located between a person's foot and a floor. The sensed force is then displayed on the visual indicator, e.g., the LCD screen 112 in the form of pounds or any other unit of force, or in terms of the total amount of an allowed force e.g., a percentage of the force allowed by the setpoint. In embodiments, the body 110 includes one or more manual input fixtures 129 e.g., the weight limit adjustment buttons 130 and 131, which allow the control unit to be configured with one or more setpoints. These input fixtures may also allow for the unit to powered on and off. In embodiments, the setpoint is predetermined and is not adjustable. In other embodiments, the manual input fixtures may include calibrated dials, touch screen inputs, and/or any combination thereof.

As shown in FIG. 5, the electronic processing unit 116, power source 118 and indication devices 112 170 and 150 are located on a circuit board 141 disposed within housing 110. Accordingly, in embodiments the indication device is integral with the medical device. In embodiments, the housing comprises one or more attachment straps dimensioned and arranged to engage the housing with the portion of the living organism.

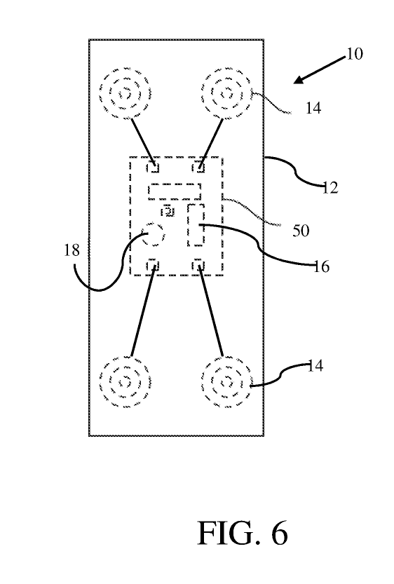

Turning to FIG. 6, in embodiments the medical device 10 comprises a housing 12 dimensioned and arranged to be disposed on, within and/or integral to an orthopedic device. In embodiments the electronic processing unit 16, power source 18 and other circuitry are located on a circuit board 50, which is in electronic communication with one or more force sensors 14. As shown in FIG. 6, in embodiments the medical treatment device comprises a plurality of force sensors 14, each in electronic communication with at least one electronic processing unit 16.

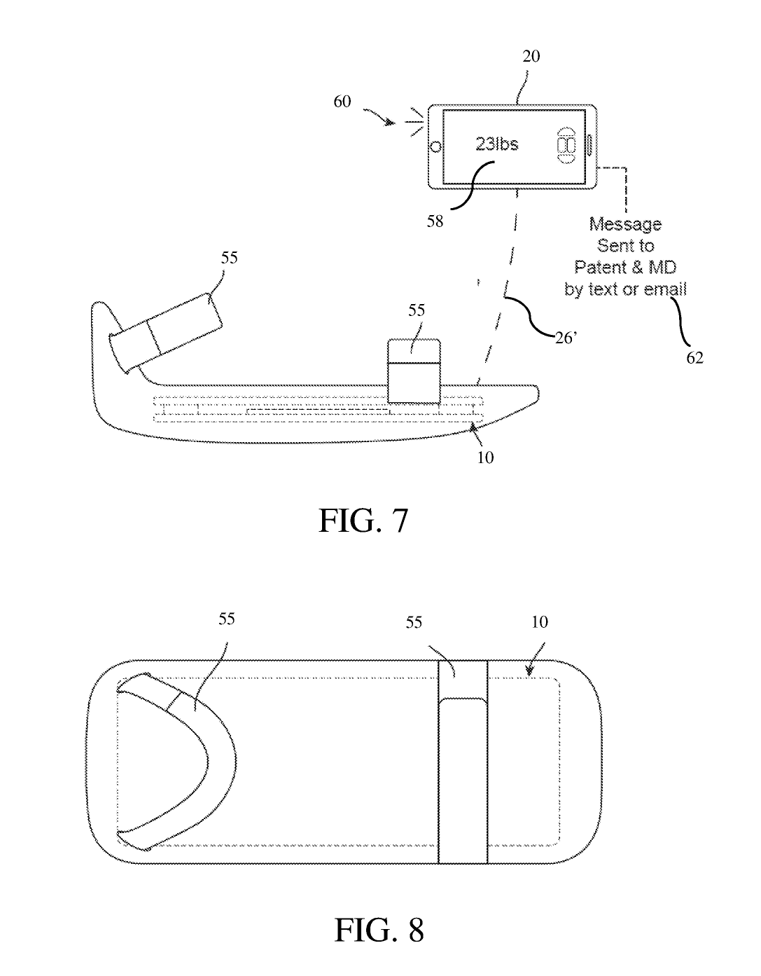

Turning to FIG. 7, in an embodiment the medical device 10 is integral to a body covering, such as the orthopedic brace, i.e., the “walking shoe” shown in FIG. 7. As shown in FIG. 7, the medical device is releasably engagable with a living organism, e.g., a human foot via the attachment straps 55 which hold the brace onto the limb. The electronic processing unit of medical device 10 (see FIG. 1) is in wireless communication with the indication device 20, which is a portable electronic device (e.g., a smart phone) running an app configured to communicate with the electronic processing unit. As is shown, force applied to the medical device results in a visual indication 58 on the display screen of the smart phone, and/or an audible and/or haptic indication 60 via the smart phone. As is also shown, in an embodiment, the portable electronic device may send a text message or email 62 to the wearer and/or to a clinician or other person or system monitoring the wearer, and/or may log the data via the internet or other wireless communication for use by the clinician.

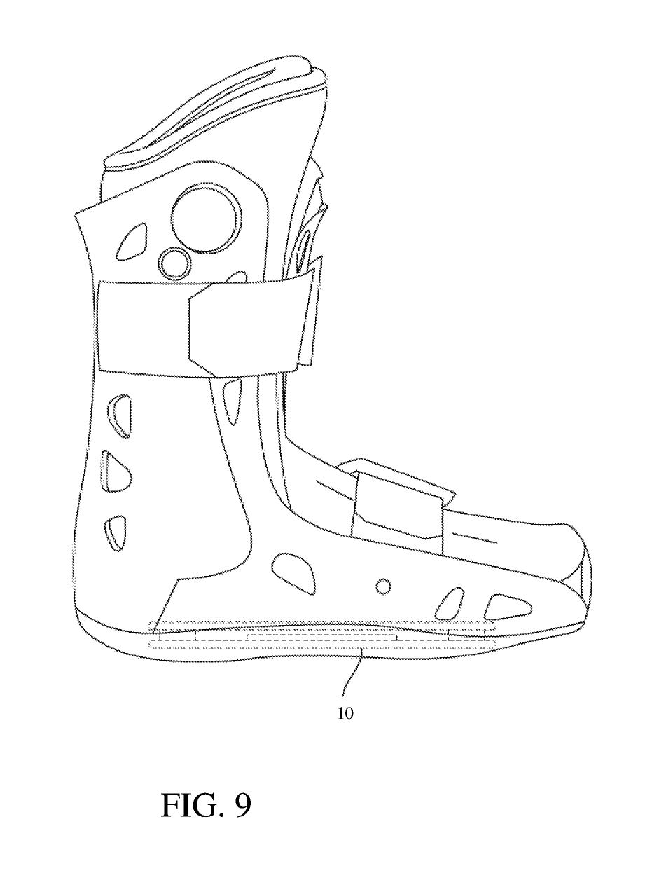

FIG. 8 shows a top view of the embodiment shown in FIG. 7.

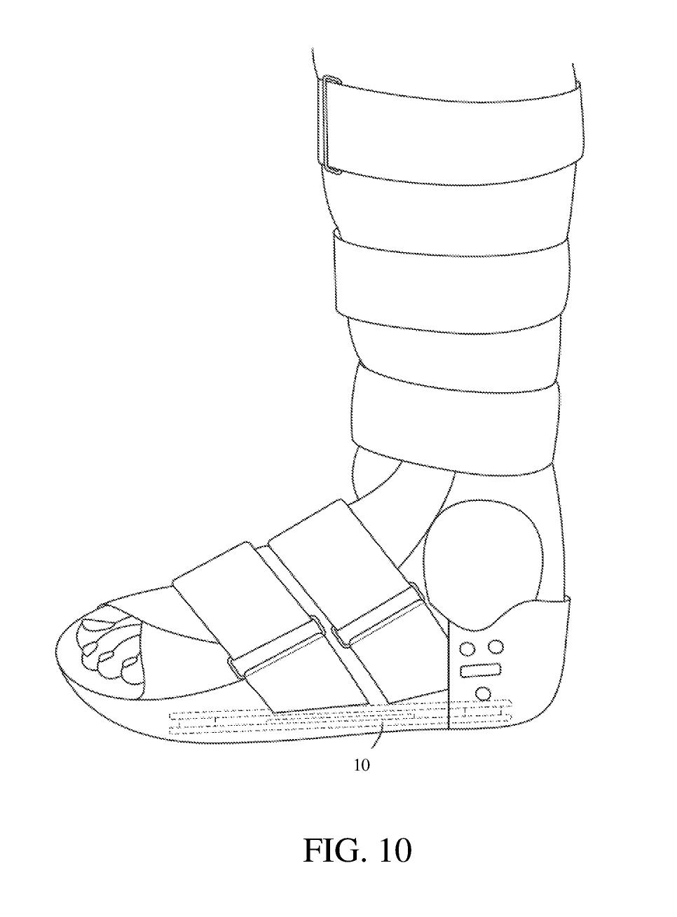

FIG. 9 shows an orthopedic device, e.g., a removable walking boot in which the medical device is integral to the boot, disposed between the inside bottom and the sole of the boot. FIG. 10 shows the orthopedic device shown in FIG. 9, disposed on a human foot.

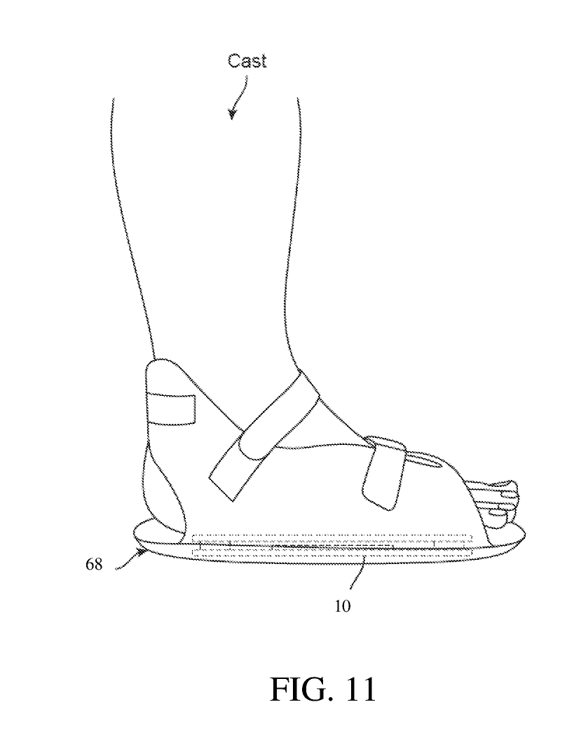

FIG. 11 shows the medical device 10 disposed in a surgical shoe 68, which is fitted over a human leg and foot wearing a plaster cast 70.

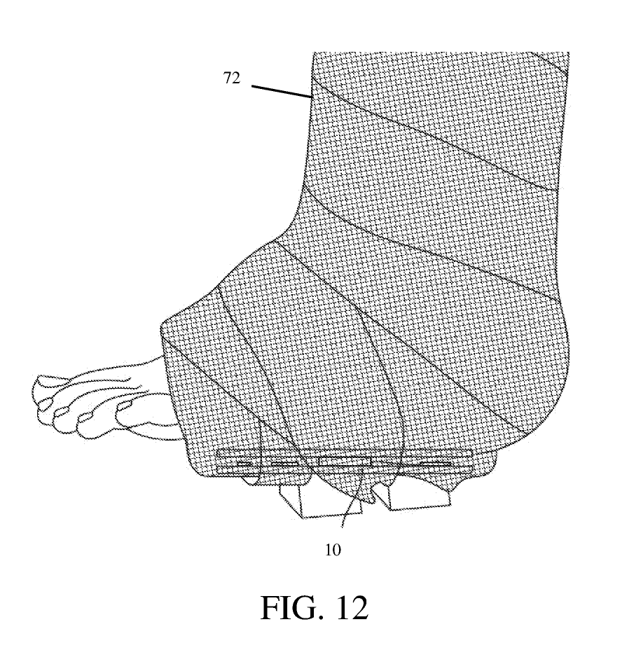

FIG. 12 shows the medical device 10 formed into the fiberglass cast 72 around a human leg and foot.

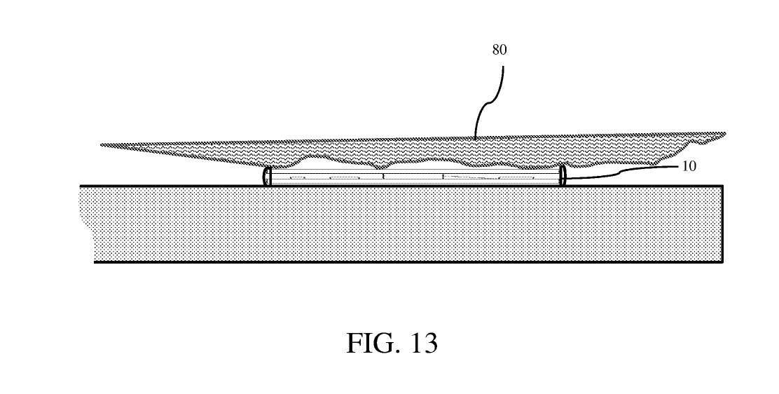

As shown in FIG. 13, in embodiments the medical treatment device 10 is disposed between the portion of the living organism 80 and an external surface 82, wherein the force sensor of the medical device 10 is arranged to measure the force applied to the portion of the living organism consistent with the formation of pressure sores, pressure ulcers, bed sores, or a combination thereof. For example, the portion of the living organism 80 may be proximate to bony areas of the human body, such as the heels, ankles, hips, tailbone, and/or those in contact with orthopedic devices, and the external surface 82 may be a bed, wheel chair seat, orthopedic brace, or other surface in which sustained pressure and/or rubbing above a selected set point or within a selected force range results in the formation of pressure sores, e.g., bedsores, pressure ulcers, decubitus ulcers, and the like.

In embodiments, a method of medical treatment comprises the steps of:

a) providing a medical treatment device according to one or more embodiments disclosed herein, e.g., comprising a housing releasably engagable with the portion of a living organism and comprising a force sensor in electronic communication with an electronic processing unit and a power source, wherein the electronic processing unit is in electronic communication with or is capable of electronic communication with an indication device; wherein the electronic processing unit is programmable to accept one or more predetermined force setpoints, and/or one or more ranges of force, and wherein the electronic processing unit actuates the indication device to provide a first visual indication, a first audible indication, or both, when an amount of force applied to the force sensor exceeds a first force setpoint, is within a first predetermined range of force, or a combination thereof;
b) programming the electronic processing unit with one or more predetermined force setpoints, and/or one or more ranges of force, and
c) engaging the medical treatment device with a portion of a living organism.

In embodiments, the method may further include recording the force applied to the force sensor over a period of time in the form of digital data, and optionally electronically retrieving these data from the electronic storage using the external data retrieval device, and/or placing the electronic processing unit in electronic communication with the portable electronic device comprising the indication device.

In embodiments, the method may include disposing the housing between a portion of the living organism and an external surface wherein pressure sores are known to form, wherein the force sensor is arranged to measure the force applied to the portion of the living organism consistent with the formation of pressure sores, pressure ulcers, bed sores, or a combination thereof, and wherein the one or more predetermined force setpoints, and/or the one or more ranges of force are selected to be in a range known to form pressure sores, pressure ulcers, bed sores, or a combination thereof. The method may further include monitoring of the pressure via wireless electronic communication.

### Embodiments Listing

Accordingly, the instant disclosure provides the following embodiments:
E1. A medical treatment device comprising:
   a housing releasably engagable with a portion of a living organism comprising a force sensor in electronic communication with an electronic processing unit and a power source;
   wherein the electronic processing unit is in electronic communication with an indication device,
   wherein the electronic processing unit is capable of electronic communication with the indication device, or a combination thereof.
E2. The medical treatment device of embodiment E1, wherein the electronic processing unit is programmable to accept one or more predetermined force setpoints, and/or one or more ranges of force, and wherein the electronic processing unit actuates the indication device to provide a first visual indication, a first audible indication, a first haptic indication, or a combination thereof, when an amount of force applied to the force sensor exceeds a first force setpoint, is within a first predetermined range of force, or a combination thereof, and optionally to actuate the indication device to provide a second visual indication, a second audible indication, a second haptic indication, or a combination thereof, when the amount of force applied to the force sensor exceeds a second force setpoint, is within a second predetermined range of force, or a combination thereof.
E3. The medical treatment device of embodiment E1 or E2, wherein the electronic processing unit includes an electronic storage which records the force applied to the force sensor in the form of digital data, and wherein these data are retrievable from the electronic storage by an external data retrieval device placed in electronic communication with the electronic processing unit.
E4. The medical treatment device of any one of embodiments E1 through E3, further comprising a temperature sensor, an acceleration sensor, a clock, or a combination thereof in electronic communication with the electronic processing unit, and wherein the electronic processing unit records the temperature, time, acceleration force, or a combination thereof in the form of digital data, and wherein these data are retrievable from the electronic storage by the external data retrieval device placed in electronic communication with the electronic processing unit.
E5. The medical treatment device of any one of embodiments E1 through E4, wherein the indication device is located on the housing, is a dedicated electronic device in wireless communication with the electronic processing unit, or a combination thereof.
E6. The medical treatment device of any one of embodiments E1 through E5, wherein the indication device comprises a program running on a portable electronic device, and wherein the electronic processing unit is in, or is capable of being placed in wired electronic communication, wireless electronic communication, or intermittent wireless electronic communication with the portable electronic device.
E7. The medical treatment device of embodiment E6, wherein the portable electronic device is a smart phone, a tablet, or a hand-held computing device.
E8. The medical treatment device of any one of embodiments E1 through E7, comprising a plurality of force sensors, each in electronic communication with at least one electronic processing unit.
E9. The medical treatment device of any one of embodiments E1 through E8, wherein the housing comprises one or more attachment straps dimensioned and arranged to engage the housing with the portion of the living organism.
E10. The medical treatment device of any one of embodiments E1 through E9 wherein the housing is integral to, or dimensioned and arranged to be disposed on or within an orthopedic device.
E11. The medical treatment device of embodiment E10, wherein the orthopedic device is selected from the group consisting of: a removable orthopedic brace, a removable orthopedic cast, an orthopedic brace, a plaster cast, a synthetic cast, a splint, an implant, a prosthesis, a wheel chair, crutches, and a combination thereof.
E12. The medical treatment device of any one of embodiments E1 through E11, wherein the housing is disposed within or is integral to a foot covering, and wherein the force sensor is arranged to measure the force applied to the foot upon contact of an outer portion of the foot covering with an external surface.
E13. The medical treatment device of any one of embodiments E1 through E11, wherein the housing is disposed within, or is integral to a hand covering, a wrist covering, or a combination thereof, and wherein the force sensor is arranged to measure the force applied to the hand, the wrist, or a combination thereof upon contact of an outer portion of the covering with an external surface.
E14. The medical treatment device of any one of embodiments E1 through E11, wherein the housing is disposed between the portion of the living organism and an external surface, and wherein the force sensor is arranged to measure the force applied to the portion of the living organism consistent with the formation of pressure sores, pressure ulcers, bed sores, or a combination thereof.
M15. A method of medical treatment comprising the steps of:
   providing a medical treatment device according to any one of embodiments E1 through E14, programming the electronic processing unit with one or more predetermined force setpoints, and/or one or more ranges of force, and engaging the medical treatment device with a portion of a living organism.
M16. A method of medical treatment comprising the steps of:
   providing a medical treatment device comprising a housing releasably engagable with the portion of a living organism and comprising a force sensor in electronic communication with an electronic processing unit and a power source, wherein the electronic processing unit is in electronic communication with or is capable of electronic communication with an indication device;
   wherein the electronic processing unit is programmable to accept one or more predetermined force setpoints, and/or one or more ranges of force, and wherein the electronic processing unit actuates the indication device to provide a first visual indication, a first audible indication, or both, when an amount of force applied to the force sensor exceeds a first force setpoint, is within a first predetermined range of force, or a combination thereof;
   programming the electronic processing unit with one or more predetermined force setpoints, and/or one or more ranges of force, and
   engaging the medical treatment device with a portion of a living organism.
M17. The method of embodiment M15 or M16, wherein the electronic processing unit includes an electronic storage which records the force applied to the force sensor in the form of digital data, and wherein these data are retrievable from the electronic storage by an external data retrieval device in electronic communication with the electronic processing unit; and further comprising recording the force applied to the force sensor over a period of time in the form of digital data, and optionally electronically retrieving these data from the electronic storage using the external data retrieval device.
M18. The method of any one of embodiments M15 through M17 wherein the indication device comprises a program running on a portable electronic device; and further comprising placing the electronic processing unit in electronic communication with the portable electronic device comprising the indication device.
M19. The method of any one of embodiments M15 through M18, wherein the housing is disposed between the portion of the living organism and an external surface, and wherein the force sensor is arranged to measure the force applied to the portion of the living organism consistent with the formation of pressure sores, pressure ulcers, bed sores, or a combination thereof, and wherein the one or more predetermined force setpoints, and/or the one or more ranges of force are in a range known to form pressure sores, pressure ulcers, bed sores, or a combination thereof.

The foregoing disclosure and description of the invention is illustrative and explanatory thereof and it can be readily appreciated by those skilled in the art that various changes in the size, shape and materials, as well as in the details of the illustrated construction or combinations of the elements described herein can be made without departing from the spirit of the invention.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

I claim:

1. A medical treatment device comprising:
a housing releasably engagable with a portion of a living organism comprising a force sensor in electronic communication with an electronic processing unit and a power source;
wherein the electronic processing unit is in electronic communication with an indication device,
wherein the electronic processing unit is capable of electronic communication with the indication device, or a combination thereof.

2. The medical treatment device of claim 1, wherein the electronic processing unit is programmable to accept one or more predetermined force setpoints, and/or one or more ranges of force, and wherein the electronic processing unit actuates the indication device to provide a first visual indication, a first audible indication, a first haptic indication, or a combination thereof, when an amount of force applied to the force sensor exceeds a first force setpoint, is within a first predetermined range of force, or a combination thereof, and optionally to actuate the indication device to provide a second visual indication, a second audible indication, a second haptic indication, or a combination thereof, when the amount of force applied to the force sensor exceeds a second force setpoint, is within a second predetermined range of force, or a combination thereof.

3. The medical treatment device according to claim 1, wherein the electronic processing unit includes an electronic storage which records the force applied to the force sensor in the form of digital data, and wherein these data are retrievable from the electronic storage by an external data retrieval device placed in electronic communication with the electronic processing unit.

4. The medical treatment device of claim 3, further comprising a temperature sensor, an acceleration sensor, a clock, or a combination thereof in electronic communication with the electronic processing unit, and wherein the electronic processing unit records the temperature, time, acceleration force, or a combination thereof in the form of digital data, and wherein these data are retrievable from the electronic storage by the external data retrieval device placed in electronic communication with the electronic processing unit.

5. The medical treatment device according to claim 1, wherein the indication device is located on the housing, is a dedicated electronic device in wireless communication with the electronic processing unit, or a combination thereof.

6. The medical treatment device according to claim 1, wherein the indication device comprises a program running on a portable electronic device, and wherein the electronic processing unit is in, or is capable of being placed in wired electronic communication, wireless electronic communication, or intermittent wireless electronic communication with the portable electronic device.

7. The medical treatment device of claim 6, wherein the portable electronic device is a smart phone, a tablet, or a hand-held computing device.

8. The medical treatment device according to claim 1, comprising a plurality of force sensors, each in electronic communication with at least one electronic processing unit.

9. The medical treatment device according to claim 1, wherein the housing comprises one or more attachment straps dimensioned and arranged to engage the housing with the portion of the living organism.

10. The medical treatment device according to claim 1 wherein the housing is integral to, or dimensioned and arranged to be disposed on or within an orthopedic device.

11. The medical treatment device of claim 10, wherein the orthopedic device is selected from the group consisting of: a removable orthopedic brace, a removable orthopedic cast, an orthopedic brace, a plaster cast, a synthetic cast, a splint, an implant, a prosthesis, a wheel chair, a bed, crutches, and a combination thereof.

12. The medical treatment device according to claim 10, wherein the housing is disposed within or is integral to a foot covering, and wherein the force sensor is arranged to measure the force applied to the foot upon contact of an outer portion of the foot covering with an external surface.

13. The medical treatment device according to claim 1, wherein the housing is disposed within, or is integral to a hand covering, a wrist covering, or a combination thereof, and wherein the force sensor is arranged to measure the force applied to the hand, the wrist, or a combination thereof upon contact of an outer portion of the covering with an external surface.

14. The medical treatment device according to claim 1, wherein the housing is disposed between the portion of the living organism and an external surface, and wherein the force sensor is arranged to measure the force applied to the portion of the living organism consistent with the formation of pressure sores, pressure ulcers, bed sores, or a combination thereof.

15. A method of medical treatment comprising the steps of:
providing a medical treatment device comprising a housing releasably engagable with the portion of a living organism and comprising a force sensor in electronic communication with an electronic processing unit and a power source, wherein the electronic processing unit is in electronic communication with or is capable of electronic communication with an indication device;
wherein the electronic processing unit is programmable to accept one or more predetermined force setpoints, and/or one or more ranges of force, and wherein the electronic processing unit actuates the indication device to provide a first visual indication, a first audible indication, or both, when an amount of force applied to the force sensor exceeds a first force setpoint, is within a first predetermined range of force, or a combination thereof;
programming the electronic processing unit with one or more predetermined force setpoints, and/or one or more ranges of force, and
engaging the medical treatment device with a portion of a living organism.

16. The method of claim 15, wherein the electronic processing unit includes an electronic storage which records the force applied to the force sensor in the form of digital data, and wherein these data are retrievable from the electronic storage by an external data retrieval device in electronic communication with the electronic processing unit; and
further comprising recording the force applied to the force sensor over a period of time in the form of digital data, and optionally electronically retrieving these data from the electronic storage using the external data retrieval device.

17. The method of claim 15, wherein the indication device comprises a program running on a portable electronic device; and further comprising placing the electronic processing unit in electronic communication with the portable electronic device comprising the indication device.

18. The method of claim 15, wherein the housing is disposed between the portion of the living organism and an external surface, and wherein the force sensor is arranged to measure the force applied to the portion of the living organism consistent with the formation of pressure sores, pressure ulcers, bed sores, or a combination thereof, and wherein the one or more predetermined force setpoints, and/or the one or more ranges of force are selected to be in a range known to form pressure sores, pressure ulcers, bed sores, or a combination thereof.
